(19) United States
(12) Patent Application Publication
(10) Pub. No.: US 2024/0315797 A1
(43) Pub. Date: Sep. 26, 2024

(54) MICRODRIVE GUIDE

(71) Applicant: TEXAS SCIENTIFIC INSTRUMENTS, LLC, BOERNE, TX (US)

(72) Inventor: DENNIS SHAUN McCARTHY, BOERNE, TX (US)

(73) Assignee: TEXAS SCIENTIFIC INSTRUNENTS, LLC, BOERNE, TX (US)

(21) Appl. No.: 18/415,783

(22) Filed: Jan. 18, 2024

Related U.S. Application Data
(60) Provisional application No. 63/491,947, filed on Mar. 23, 2023.

(51) Int. Cl. A61B 90/11; A61B 90/10; A61N 1/05

(52) U.S. Cl. CPC A61B 90/11; A61B 2090/103; A61N 1/0539

(57) ABSTRACT

A life science precision neuroelectrophysiology instrument system to hold precision instruments such as canulae, measuring instruments, microsyringe needles and electrodes, and provide finely adjustable control of the delivery of said instruments to a surgical site.

### CROSS-REFERENCE

This application claims the benefit of U.S. Provisional Application No. 63/491,947, filed Mar. 23, 2023, which application is incorporated herein by reference as if reproduced in full below.

### BACKGROUND

The present disclosure is directed to instruments useful in fields such as intracerebral infusion or neuroelectrophysiology where microsurgical work is performed.

In particular this disclosure is directed to a precision instrument guide utilized to hold precision instruments such as guide canulae, measuring instruments, microsyringe needles and electrodes, and provide finely adjustable control of the delivery of said instruments to a surgical site.

### SUMMARY

Neuroelectrophysiology is the study of electrical properties of biological cells and tissues within the nervous system. It is used to determine how neuronal disorders happen by looking at the individual's brain activity. Neuroelectrophysiology is the electrophysiology of neurons.

Neuroelectrophysiology is an old science, dating to the 18th century when electrical activity in nerves was discovered. Such discoveries have led to a variety of neurophysiological techniques, ranging from basic neuroscience to clinical applications. These clinical applications allow assessment of complex neurological functions such as (but not limited to) sensory perception (vision, hearing, somatosensory function), and muscle function. The ability to use similar techniques in both human and animal models increases the ability to perform mechanistic research to investigate neurological problems. Good animal to human homology of many neurophysiological systems facilitates interpretation of data to provide cause-effect linkages to epidemiological findings.

The science and surgical techniques used in this field, as in all areas of Life Science, continues to evolve, most frequently in the laboratory and veterinary medical fields where new techniques and instruments are developed and tested for further application and advancements in other areas of life sciences.

Surgical work performed in and around the brain, or anywhere within the nervous system, must be precise. As such, instruments must be designed with a degree of accuracy and be capable of delivering their payload with precision.

Provided herein is a life sciences precision medical instrument guide system comprising: a base plate having a vertical oriented protrusion thereon with a port extending through the protrusion and plate, a guide cannula, an electrode and a port plug.

In some embodiments, the precision medical instrument guide system further comprises a microsyringe needle, a micrometer or other measuring instrument; and a dummy cannula.

In some embodiments, the precision medical instrument guide system further comprises two or more fixation features on the base plate comprising: holes, screws, nails, pins, spikes, protrusions or a combination thereof, wherein said fixation features are configured to provide for fixation of the base plate to a subject's boney anatomy.

In some embodiments, the precision medical instrument guide system further comprises precision threads located on the protrusion, the guide cannula, the electrode, the dummy cannula, and the port plug, configured to provide precision depth adjustment.

In some embodiments, the precision medical instrument guide system further comprises precision threads located on the microsyringe needle, and the micrometer or other measuring instrument.

In some embodiments of the precision medical instrument guide system, said precision threads are located on an exterior diameter of any component of the system; an interior diameter of any component of the system or both an interior and exterior diameter of any component of the system.

In some embodiments of the precision medical instrument guide system, said precision threads comprise, an imperial fine thread series, a metric fine or extra fine thread series, an AJS Series, a Thury metric screw thread, a Löwenherz thread, a coarse thread series or a combination thereof.

In some embodiments of the precision medical instrument guide system, said base plate is further configured for fixation to a subject's boney anatomy with a medical grade adhesive.

In some embodiments, the precision medical instrument guide system an inferior surface of the baseplate is either flat or comprises a concave surface configured to closely conform to a boney anatomy of a skull.

In some embodiments of the precision medical instrument guide system, said components can be manufactured using a variety of processes comprising machining, injection molding, casting, additive manufacturing, or combinations thereof.

In some embodiments of the precision medical instrument guide system said components can be manufactured using a variety of biocompatible medical grade materials comprising stainless steels, polytetrafluoroethylene (PTFE)/Teflon™, thermoplastics, thermosets, elastomers or a combination thereof.

In some embodiments of the precision medical instrument guide system said baseplate material comprises elastic behavior and plastic deformation properties that are to the left of the materials fracture point on a stress/strain curve, where strain is on the x-axis, when the baseplate is flexed to conform to the boney anatomy of the skull.

In some embodiments, the precision medical instrument guide system is configured for use in multiple areas of Life Sciences that relate to a living being; animal or human.

In some embodiments, the precision medical instrument guide system is configured for use in the veterinary medicine arts.

Provided herein is a precision medical instrument guide comprising a base plate having a port therethrough, a vertically oriented protrusion configured to extend from the base plate, two or more fixation features spaced about the base plate, a port extending through the protrusion, configured to align with the port of the plate and threads located on the protrusion configured to receive mating threads from another instrument configured to adapt to the guide.

In some embodiments of the precision medical instrument guide, the threads are located within the port of the protrusion; located on the exterior of the protrusion or located both within the port and on the exterior of the protrusion.

In some embodiments of the precision medical instrument guide, the two or more fixation features comprise holes, screws, nails, pins, spikes, protrusions, or a combination thereof, wherein said fixation features are configured to provide for fixation of the base plate to a subject's boney anatomy.

In some embodiments of the precision medical instrument guide, said base plate is further configured for attachment to a subject's boney anatomy with a medical grade adhesive.

In some embodiments of the precision medical instrument guide, said base plate is further configured for attachment to a subject's exposed skull.

In some embodiments of the precision medical instrument guide, the base plate and the protrusion are configured as a single unit or as a multi-piece assembly.

In some embodiments of the precision medical instrument guide, said precision threads are configured to receive attachments comprising a threaded guide cannula, a threaded electrode, a threaded dummy cannula, or a threaded port plug.

In some embodiments of the precision medical instrument guide, said precision threads are further configured to receive attachments comprising a microsyringe needle; and a micrometer or other measuring instrument.

In some embodiments of the precision medical instrument guide, said precision threads comprise an imperial fine thread series, a metric fine or extra fine thread series, an AJS thread series, a Thury metric screw thread series, a Löwenherz thread series or a coarse thread series.

In some embodiments of the precision medical instrument guide, said components can be manufactured using a variety of processes comprising machining, injection molding, casting, additive manufacturing; or combinations thereof.

In some embodiments of the precision medical instrument guide, said components can be manufactured using a variety of biocompatible medical grade materials comprising stainless steels, polytetrafluoroethylene (PTFE)/Teflon™, thermoplastics, thermosets, elastomers, or a combination thereof.

In some embodiments of the precision medical instrument guide, wherein said baseplate material comprises elastic behavior and plastic deformation properties that are to the left of the materials fracture point on a stress/strain curve, where strain is on the x-axis, when the baseplate is flexed to conform to the boney anatomy of the skull.

In some embodiments, the precision medical instrument guide system is configured for use in multiple areas of Life Sciences.

In some embodiments, the precision medical instrument guide is configured for use in the veterinary medicine arts.

Provided herein is a precision medical instrument system comprising a base plate having a vertical protrusion thereon with a port extending through the protrusion and plate, a guide cannula, an electrode, a dummy cannula and a port plug, wherein said system is configured for intracerebral infusion or neuro-electrophysiology procedures where microsurgical work is performed.

In some embodiments, the precision medical instrument system further comprises a microsyringe needle; and a micrometer or other measuring instrument.

In some embodiments, the precision medical instrument system further comprises two or more fixation features spaced about the base plate to provide for fixation of the base plate to a subject's boney anatomy.

In some embodiments, the precision medical instrument system further comprises threads located on the protrusion, the guide cannula, the microsyringe needle, the electrode, the micrometer or other measuring instrument, the dummy cannula and the port plug, wherein said threads are configured to provide precision depth adjustment.

In some embodiments of the precision medical instrument system said threads are located on an exterior diameter of any component of the system; an interior diameter of any component of the system or both an interior and exterior diameter of any component of the system.

In some embodiments of the precision medical instrument system said threads comprise an imperial fine thread series, a metric fine or extra fine thread series, an AJS thread series, a Thury metric screw thread series, a Löwenherz thread series, a coarse thread series or a combination thereof.

In some embodiments of the precision medical instrument system, said base plate is further configured for attachment to a subject's boney anatomy with a medical grade adhesive for additional fixation.

In some embodiments of the precision medical instrument system, an inferior surface of the baseplate is either flat; or comprises a concave surface configured to closely conform to a boney anatomy of a skull.

In some embodiments of the precision medical instrument system, said components can be manufactured using a variety of processes comprising machining, injection molding, casting, additive manufacturing or combinations thereof.

In some embodiments of the precision medical instrument system, said components can be manufactured using a variety of biocompatible medical grade materials comprising stainless steels, polytetrafluoroethylene (PTFE)/Teflon™, thermoplastics, thermosets, elastomers or a combination thereof.

In some embodiments of the precision medical instrument system, said baseplate material comprises elastic behavior and plastic deformation properties that are to the left of the materials fracture point on a stress/strain curve, where strain is on the x-axis, when the baseplate is flexed to conform to the boney anatomy of the skull.

In some embodiments, the precision medical instrument guide system is configured for use in multiple areas of Life Sciences.

In some embodiments, the precision medical instrument system is configured for use in the veterinary medicine arts.

Additional aspects and advantages of the present disclosure will become readily apparent to those skilled in this art from the following detailed description, wherein only exemplary embodiments of the present disclosure are shown and described, simply by way of illustration of the several modes or best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a representative ISO view of a first version of the nut plate guide;

FIG. 1B is a representative top view of a first version of the nut plate guide;

FIG. 1C is a representative side view of a first version of the nut plate guide;

FIG. 1D is a representative end view of a first version of the nut plate guide;

FIG. 1E is a representative cross-section view of a first version of the nut plate guide taken through the center of the vertical oriented protrusion;

FIG. 2A is a representative ISO view of a second version of the nut plate guide;

FIG. 2B is a representative top view of a second version of the nut plate guide;

FIG. 2C is a representative side view of a second version of the nut plate guide;

FIG. 2D is a representative bottom view of a second version of the nut plate guide;

FIG. 2E is a representative end view of a second version of the nut plate guide;

FIG. 2F is a representative cross-section view of a second version of the nut plate guide taken through the center of the vertical oriented protrusion;

FIG. 3A is a representative ISO view of a third version of the nut plate guide;

FIG. 3B is a representative top view of a third version of the nut plate guide;

FIG. 3C is a representative side view of a third version of the nut plate guide;

FIG. 3D is a representative end view of a third version of the nut plate guide;

FIG. 3E is a representative cross-section view of a third version of the nut plate guide taken through the center of the vertical oriented protrusion;

FIG. 4 is representative ISO view a representative threaded guide cannula;

FIG. 5A is a representative side view of the threaded guide cannula assembly, with the guide cannula fully inserted into the first version of the nut plate guide;

FIG. 5B is a representative side view of the threaded guide cannula assembly, with the guide cannula inserted approximately one half of its depth into the first version of the nut plate guide;

FIG. 5C is a representative side view of the threaded guide cannula assembly, with the guide cannula inserted approximately one quarter of its depth into the first version of the nut plate guide;

FIG. 6 is a representative ISO view of a cannula cap;

FIG. 7A is a representative ISO view of a cannula cap inserted over a version of the threaded guide cannula assembly;

FIG. 7B is a representative ISO view of a cannula cap inserted over one version of the precision nut guide;

FIG. 8A is an ISO view of a representative threaded electrode;

FIG. 8B is a representative end view of a threaded electrode of FIG. 8A illustrating the representative contact points for receiving an electrode cable;

FIG. 9 is a representative ISO view of an electrode threaded into the second version of the nut plate guide;

FIG. 10 is an illustrative ISO view of a representative electrode cable configured for connection to the end of the threaded electrode illustrated in FIG. 8B.

The foregoing and other features of the present disclosure will become apparent from the following description and appended claims, taken in conjunction with the accompanying drawings. Understanding that these drawings depict only several embodiments in accordance with die disclosure and are, therefore, not to be considered limiting of its scope, the disclosure will be described with additional specificity and detail through use of the accompanying drawings.

### DETAILED DESCRIPTION

The present device will now be described more fully hereinafter with reference to the accompanying drawings which illustrate embodiments of the pipeline pig. This pipeline pig may, however, be embodied in many different forms and should not be construed as limited to the illustrated embodiments set forth herein. Rather, these embodiments are provided so that this disclosure will be thorough and complete, and will fully convey the scope of the device to those skilled in the art.

The following description of the exemplary embodiments refers to the accompanying drawings. The following detailed description does not limit the invention. Instead, the scope of the invention is defined by the appended claims.

Reference throughout the disclosure to “an exemplary embodiment,” “an embodiment.” or variations thereof means that a particular feature, structure, or characteristic described in connection with an embodiment is included in at least one embodiment of the subject matter disclosed. Thus, the appearance of the phrases “in an exemplary embodiment,” “in an embodiment,” or variations thereof in various places throughout the disclosure is not necessarily referring to the same embodiment. Further, the particular features, structures or characteristics may be combined in any suitable manner in one or more embodiments,

As used herein, and unless otherwise specified, the phrase “life science” or “life sciences” refers to anything that relates to a living being—animal or human—and requires something being taken from or given to that being. This could include technologies in the biotech, Al and healthcare spaces, but not all technologies in those spaces qualify. For instance, a diagnostic company that tests blood would qualify as life sciences, but an app that measures heart rates would not—that's simply a health technology.

Life sciences encompasses hard scientific development with physical products, including pharmaceuticals, therapeutics, diagnostics, medical devices and instruments, and other products that are designed to treat or aid in the treatment of patients. The life sciences sector spans different interests and markets, including academic research, pharmaceuticals, biotechnology, medical devices, diagnostics and the ultimate beneficiary of their scientific pursuits: patients.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%1, %, 0.5%, 0.1%, or 0.05% of a given value or range. In certain embodiments, the term “about” or “approximately” means within 40.0 mm, 30.0 mm, 20.0 mm, 10.0 mm 5.0 mm 1.0 mm, 0.9 mm, 0.8 mm, 0.7 mm, 0.6 mm, 0.5 mm, 0.4 mm, 0.3 mm, 0.2 mm or 0.1 mm of a given value or range. In certain embodiments, the term “about” or “approximately” means within 5.0 kg, 2.5 kg, 1.0 kg, 0.9 kg, 0.8 kg, 0.7 kg, 0.6 kg, 0.5 kg, 0.4 kg, 0.3 kg, 0.2 kg or 0.1 kg of a given value or range, including increments therein. In certain embodiments, the term “about” or “approximately” means within 1 hour, within 45 minutes, within 30 minutes, within 25 minutes, within 20 minutes, within 15 minutes, within 10 minutes, within 5 minutes, within 4 minutes, within 3 minutes within 2 minutes, or within 1 minute. In certain embodiments, the term “about” or “approximately” means within 20.0 degrees, 15.0 degrees, 10.0 degrees, 9.0 degrees, 8.0 degrees, 7.0 degrees, 6.0 degrees, 5.0 degrees, 4.0 degrees, 3.0 degrees, 2.0 degrees, 1.0 degrees, 0.9 degrees, 0.8 degrees, 0.7 degrees, 0.6 degrees, 0.5 degrees, 0.4 degrees, 0.3 degrees, 0.2 degrees, 0.1 degrees, 0.09 degrees. 0.08 degrees, 0.07 degrees, 0.06 degrees, 0.05 degrees, 0.04 degrees, 0.03 degrees, 0.02 degrees or 0.01 degrees of a given value or range, including increments therein.

As used herein, and unless otherwise specified, the term “additive manufacturing” means or refers to the process of creating a part by incrementally building it up via the addition of material. This material can be metal, ceramic, plastic, photopolymer, or even food! ISO/ASTM has categorized all the diverse types of additive manufacturing technologies into seven categories. Examples thereof include: Binder Jetting; Powder Bed Fusion; Directed Energy Deposition; Material Jetting; Sheet Lamination; Material Extrusion; and VAT Photopolymerization.

As used herein, and unless otherwise specified, the term “plurality”, and like terms, refers to a number (of things) comprising at least one (thing), or greater than one (thing), as in “two or more” (things), “three or more” (things), “four or more” (things), etc.

As used herein, the terms “connected”, “operationally connected”, “coupled”, “operationally coupled”, “operationally linked”, “operably connected”, “operably coupled”, “operably linked,” and like terms, refer to a relationship (mechanical, linkage, coupling, etc.) between elements whereby operation of one element results in a corresponding, following, or simultaneous operation or actuation of a second element. It is noted that in using said terms to describe inventive embodiments, specific structures or mechanisms that link or couple the elements are typically described. However, unless otherwise specifically stated, when one of said terms is used, the term indicates that the actual linkage or coupling may take a variety of forms, which in certain instances will be readily apparent to a person of ordinary skill in the relevant technology.

As used herein, the terms “comprises”, “comprising”, or any other variation thereof, are intended to cover a nonexclusive inclusion, such that a process, method, article, or apparatus that comprises a list of elements does not include only those elements but may include other elements not expressly listed or inherent to such process, method, article, or apparatus.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms “user”, “subject” or “patient” are used interchangeably. As used herein, the terms “subject” and “subjects” refers to an animal (e.g., birds, reptiles, and mammals), preferably a mammal including a primate (e.g., a monkey, chimpanzee, and a human) (and a non-primate (e.g., a camel, donkey, zebra, cow, pig, horse, cat, dog, rat, and mouse). In certain embodiments, the mammal is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old, 95 to 100 years old or over 100 years old. In one preferred embodiment, the subject or patient is a pig. In certain embodiments, the pig is 0 to 6 months old, 6 to 12 months old, 1 to 5 years old, 5 to 10 years old or 10 to 15 years old. The natural lifespan of a pig is 10-15 years. In another preferred embodiment, the subject or patient is a human. In certain embodiments, the human is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 40 to 60 years old, 60 to 70 years old, 70 to 80 years old, 80 to 90 years old, or 90 to 110 years old. The upper limit of the natural lifespan or maximum lifespan of a human is generally accepted to be about 125 years.

As used herein, and unless otherwise specified, the term “anterior” can refer to human anatomy and means the front surface of the body; often used to indicate the position of one structure relative to another, that is, situated nearer the front part of the body. Alternately, it may also refer in a similar fashion to an apparatus or structure.

As used herein, and unless otherwise specified, the term “posterior” can refer to human anatomy and means the back surface of the body; Often used to indicate the position of one structure relative to another, that is, nearer the back of the body. Alternately, it may also refer in a similar fashion to an apparatus or structure.

As used herein, and unless otherwise specified, the term “superior” can refer to human anatomy and means situated nearer the vertex of the head in relation to a specific reference point; opposite of inferior. It may also mean situated above or directed upward. Alternately, it may also refer in a similar fashion to an apparatus or structure.

As used herein, and unless otherwise specified, the term “inferior” can refer to human anatomy and means situated nearer the soles of the feet in relation to a specific reference point; opposite of superior. It may also mean situated below or directed downward. Alternately, it may also refer in a similar fashion to an apparatus or structure.

As used herein, and unless otherwise specified, the term “medial” can refer to human anatomy and means situated toward the median plane or midline of the body. Alternately, it may also refer in a similar fashion to an apparatus or structure.

As used herein, and unless otherwise specified, the term “lateral” can refer to human anatomy and means denoting a position farther from the median plane or midline of the body or a structure. It may also mean “pertaining to a side”. Alternately, it may also refer in a similar fashion to an apparatus or structure.

As used herein, the term “proximity” means nearness in space or relationship, but not excluding the potential to be touching. Proximity is also alternatively meant to mean that one thing may be so close to another thing as to be “in direct or nearly direct contact” (in proximity) with another thing along some point. To “place something in proximity” is also meant to mean that items are “paired” or “mated together” either in their paired function or at some point of contact.

As used herein, and unless otherwise specified, the term “vertically oriented” and similar terms mean; generally perpendicular to, at, or near, right angles to a horizontal plane; in a direction or having an alignment such that the top of a thing is above the bottom. In certain embodiments, the term “vertically oriented” means within ±20.0 degrees, ±15.0 degrees, ±10.0 degrees, ±9.0 degrees, ±8.0 degrees, ±7.0 degrees, ±6.0 degrees, ±5.0 degrees, ±4.0 degrees, ±3.0 degrees, ±2.0 degrees, ±1.0 degrees, ±0.9 degrees, ±0.8 degrees, ±0.7 degrees, +0.6 degrees, ±0.5 degrees, ±0.4 degrees, ±0.3 degrees, ±0.2 degrees or ±0.1 degrees of a given value or range, including increments therein.

As used herein, and unless otherwise specified, the term “horizontally oriented” and similar terms mean; generally perpendicular to, at, or near, right angles to a vertical plane; in a direction or having an alignment such that the top of a thing is generally on, or near the same plane as the bottom, both being parallel or near parallel to the horizon. In certain embodiments, the term “horizontally oriented” means within +20.0 degrees, ±15.0 degrees, +10.0 degrees, ±9.0 degrees, ±8.0 degrees, ±7.0 degrees, ±6.0 degrees, ±5.0 degrees, ±4.0 degrees, ±3.0 degrees, ±2.0 degrees, ±1.0 degrees, ±0.9 degrees, ±0.8 degrees, ±0.7 degrees, +0.6 degrees, ±0.5 degrees, ±0.4 degrees, ±0.3 degrees, ±0.2 degrees or +0.1 degrees of a given value or range, including increments therein.

As used herein, and unless otherwise specified, the term “substantially perpendicular” and similar terms mean generally at or near 90 degrees to a given line, or surface or to the ground. In certain embodiments, the term “substantially perpendicular” means within +20.0 degrees, ±15.0 degrees, ±10.0 degrees, ±9.0 degrees, ±8.0 degrees, ±7.0 degrees, ±6.0 degrees, ±5.0 degrees, ±4.0 degrees, ±3.0 degrees, ±2.0 degrees, ±1.0 degrees, ±0.9 degrees, +0.8 degrees, ±0.7 degrees, ±0.6 degrees, ±0.5 degrees, ±0.4 degrees, ±0.3 degrees, ±0.2 degrees or +0.1 degrees of a given value or range, including increments therein.

As used herein, and unless otherwise specified, the term “precision threads” means a thread form having an imperial fine thread series, (UNF/UNRF/UNEF), or metric fine or extra fine thread series (M size x pitch) commonly used in precision applications. It may also be defined as any fine or very fine pitch thread, Because of the larger tensile stress areas, they have high tension strength. Precision threads, as used herein, also comprise extra fine and superfine threads, with very fine pitch threads because they have greater minor diameters than course threads, have a greater cross-sectional area (and therefore greater load-carrying capability) for the same nominal diameter; and they are more resistant to coming loose from vibration. Other examples of fine and very fine thread pitches include: AJS Series patented threads, Löwenherz thread, (a largely obsolete metric thread form); and the Thury metric screw thread (alternatively called the Filiére Suisse, FS, screw thread).

As used herein, and unless otherwise specified, combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C.

Provided herein is a precision medical instrument guide system comprising: the precision guide having a baseplate with a vertical oriented protrusion thereon having a port extending through the protrusion and plate, an external thread and two or more fixation features. Other components of the system comprise a guide cannula; an electrode; a dummy cannula; and a port plug.

Additional components such as a microsyringe needle and a micrometer or other measuring instrument may also be included in such a system to round out the complement of precision components a user may desire in their system of choice.

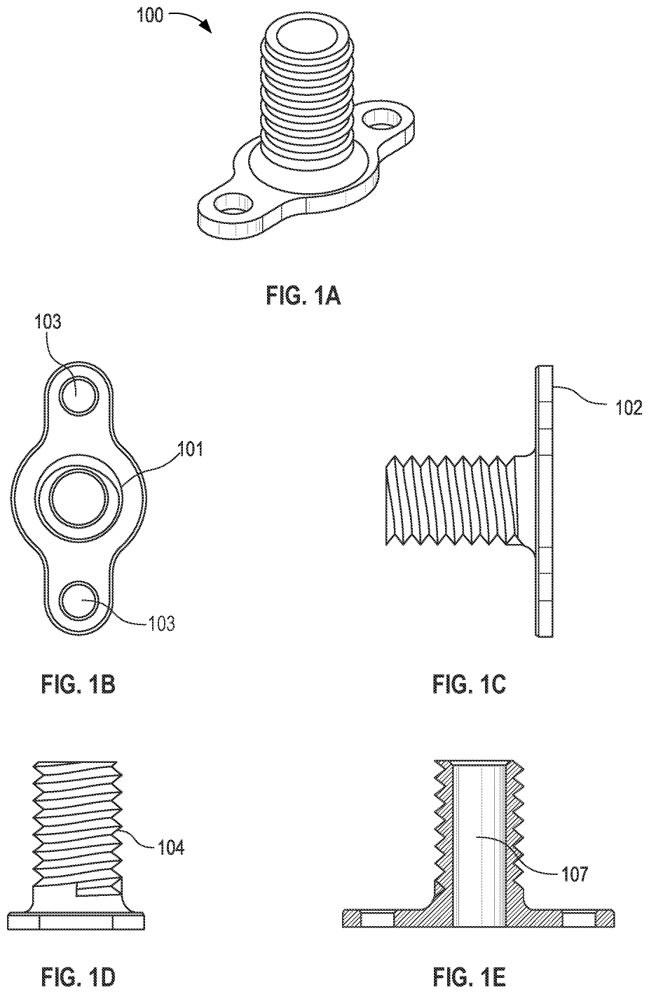

Referring now to FIGS. 1A-1E, is a first version of the precision guide 100, having a baseplate 102 with a vertically oriented protrusion 101 extending perpendicularly from the base plate. In this particular embodiment, precision threads 104 are configured on the exterior of the protrusion 101 and a thru-hole/port 107 extends through the protrusion and the base plate. The baseplate itself, comprises two or more holes 103 through which fixation features such as screws 108 (not shown) may be inserted and used to affix the base plate and protrusion to the prepared scull of a subject. Once affixed to the skull of the subject, precision instruments may be threaded onto the protrusion such as a drill guide (not shown) to pilot a drill through the skull of the subject to create an entry hole. Once an entry hole is completed and a drill guide is removed, other precision instruments, such as those described later herein, may be threaded onto/into the precision guide 100 to carry out the intended procedure.

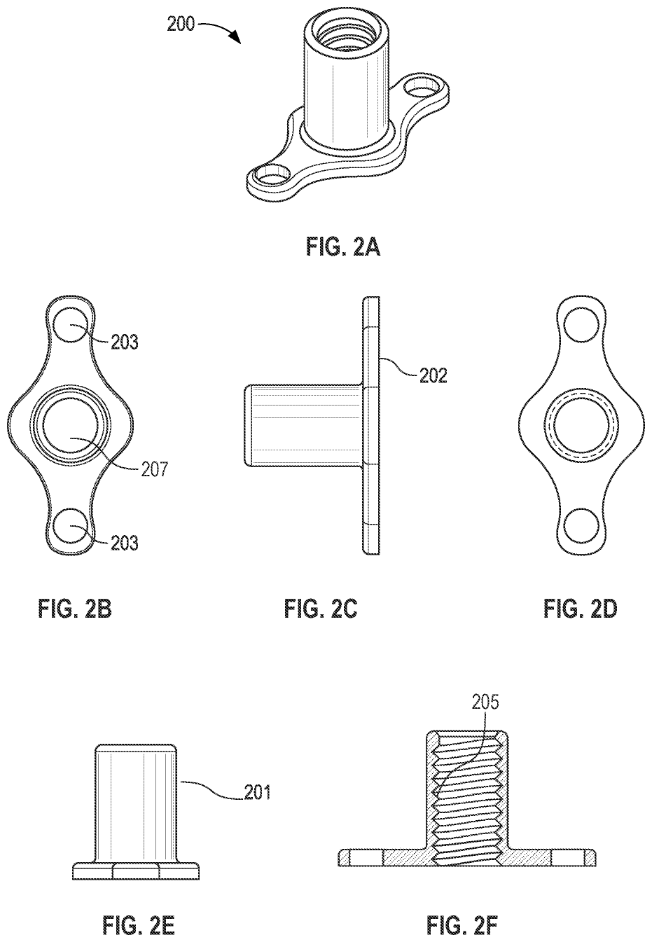

Referring now to FIGS. 2A-2F, is a second version of the precision guide 200, having a baseplate 202, also having a vertically oriented protrusion 201 extending perpendicularly from the base plate. However, different from the first version, this particular embodiment comprises precision threads 204 configured on the interior of the protrusion within the thru-hole 207 extending through the entire protrusion and the base plate. The baseplate itself, once again comprises two or more holes 203 through which fixation features such as screws 108 (not shown) may be inserted and used to affix the base plate and protrusion to the prepared scull of a subject. Once affixed to the skull of the subject, precision instruments may be threaded into the protrusion such as a drill guide to pilot a drill through the skull of the subject to create an entry hole, so that the other precision components of the medical instrument guide system can easily be attached to the precision guide and enter the brain of the subject, once the drill guide is removed.

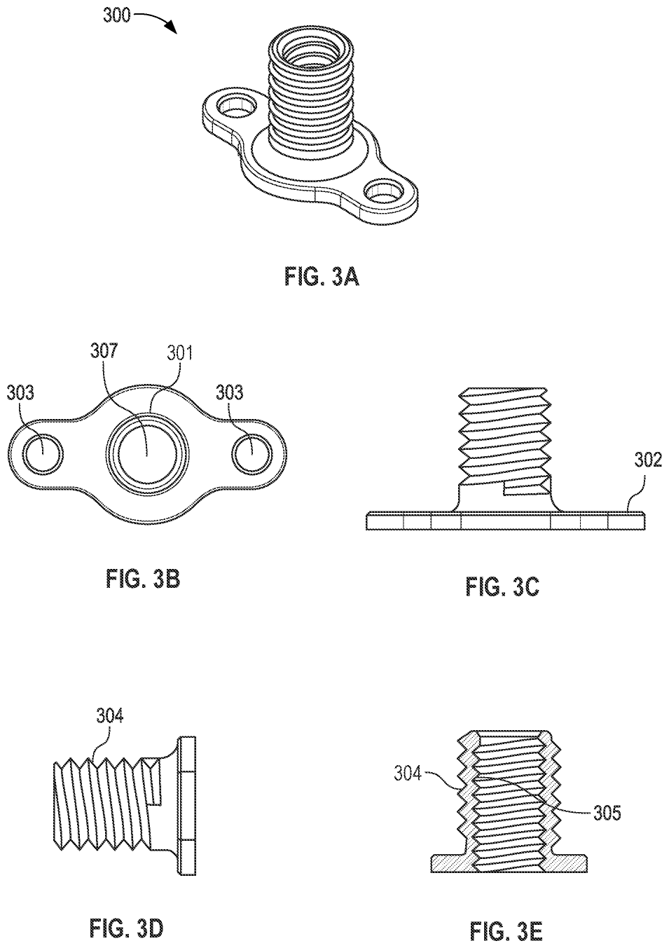

Referring now to FIGS. 3A-3E, is a third version of the precision guide 300, having a baseplate 302, also having a vertically oriented protrusion 301 extending perpendicularly from the base plate. However, different from the first and second versions, this particular embodiment comprises, precision threads 304 configured on the external diameter and another set of precision threads 305 configured on the interior of the protrusion within the thru-hole 307 extending through the entire protrusion and the base plate. The baseplate itself, once again comprises two or more holes 303 through which fixation features such as screws 108 (not shown) may be inserted and used to affix the base plate and protrusion to the prepared scull of a subject. Once affixed to the skull of the subject, precision instruments may be threaded into and onto the protrusion such as a drill guide to pilot a drill through the skull of the subject, as well as other components of the medical instrument guide system. It should be noted that the fixation features, typically screws 108, are commonly manufactured with a medical grade stainless steel. However, it may be prudent to run secondary procedures for verification of placements of the cannula or electrode involving x-rays, MRI, or microwaves. In these situations, the screws used for fixation are ideally manufactured from an alternate material such as Nylon, for example, to avoid refractory shadows on films or cause the screws to be pulled out (from the MRI).

In any one of the three versions of precision guide 100, 200, 300, shown and described herein, each component (baseplate 102, 202, 302 and protrusion 101, 201, 301) may be made as a single unit that is put together and permanently assembled or as a single assembled unit.

Further, it should be understood by one skilled in the art that any one of the three versions of precision guide shown and described herein, can be manufactured using a variety of processes comprising machining, welding, injection molding, casting, additive manufacturing, or combinations thereof. In general, additive manufacturing (AM) for many polymers can include vat polymerization (stereolithography), powder bed fusion (SLS), material and binder jetting (inkjet and aerosol 3D printing), sheet lamination (LOM), extrusion (FDM, 3D dispensing, 3D fiber deposition, and 3D plotting) and 3D bioprinting. The range of polymers used in AM encompasses thermoplastics, thermosets, elastomers, hydrogels, functional polymers, polymer blends, composites and biological systems.

Further still, one skilled in the art would also recognize that any one of the three versions of precision guide, threaded cannula, the electrode and cannula cap, shown and described herein, can be manufactured using a variety of biocompatible medical grade materials comprising stainless steels, polytetrafluoroethylene (PTFE)/Teflon™, thermoplastics, thermosets, elastomers, or a combination thereof.

Also, one skilled in the medical arts would recognize that the inferior surface of the baseplate can be either flat or comprise a concave surface configured to closely conform to a boney anatomy of a subject skull.

Finally, one of shill in the engineering arts, would also recognize the benefits of producing the baseplate from a material comprising elastic behavior and plastic deformation properties that are to the left of the materials fracture point on a stress/strain curve, where strain is on the x-axis, when the baseplate is flexed to conform to the boney anatomy of the skull. In other words, it will not fracture when it is flexed and fixed to the skull.

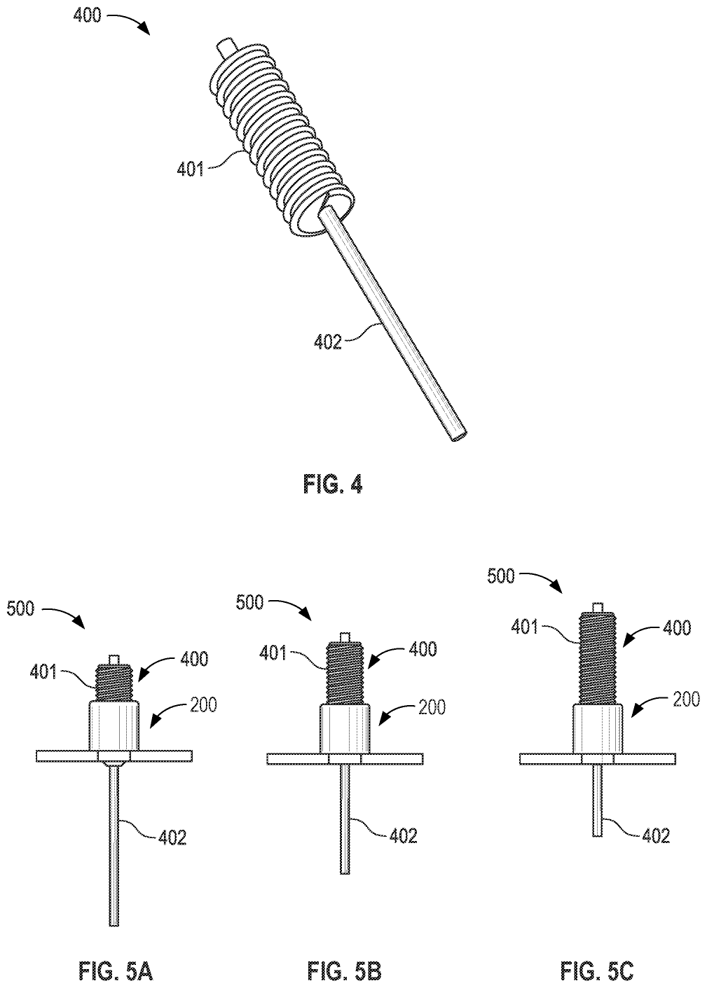

Referring now to FIG. 4, is a representative example of a threaded guide cannula 400. The representative threaded guide cannula comprises a cannula 402 with a thru-hole and a threaded outer component 401 on the approximate upper half, having a precision thread matching the thread 205, 305 on the internal diameter 207, 307 of the protrusion 201, 301 of the precision guide, as shown in the second and third versions of the precision guide 200, 300. The typical cannula for this instrument is essentially equivalent in internal and external diametral dimension to 22-26 gage hypodermic tubing.

A nearly identical variation of the threaded guide cannula comprises a solid cannula—no thru-hole—(not shown) and a threaded outer component 401 on the approximate upper half, having a precision thread matching the thread 205, 305 on the internal diameter 207, 307 of the protrusion 201, 301 of the precision guide, as shown in the second and third versions of the precision guide 200, 300. The typical solid cannula for this instrument is essentially equivalent in external diametral dimension to 22-26 gage hypodermic tubing and would be used in place of the thru-hole cannula between subsequent procedures, in order to maintain the original depth and diameter of the surgical hole produced during the original procedure.

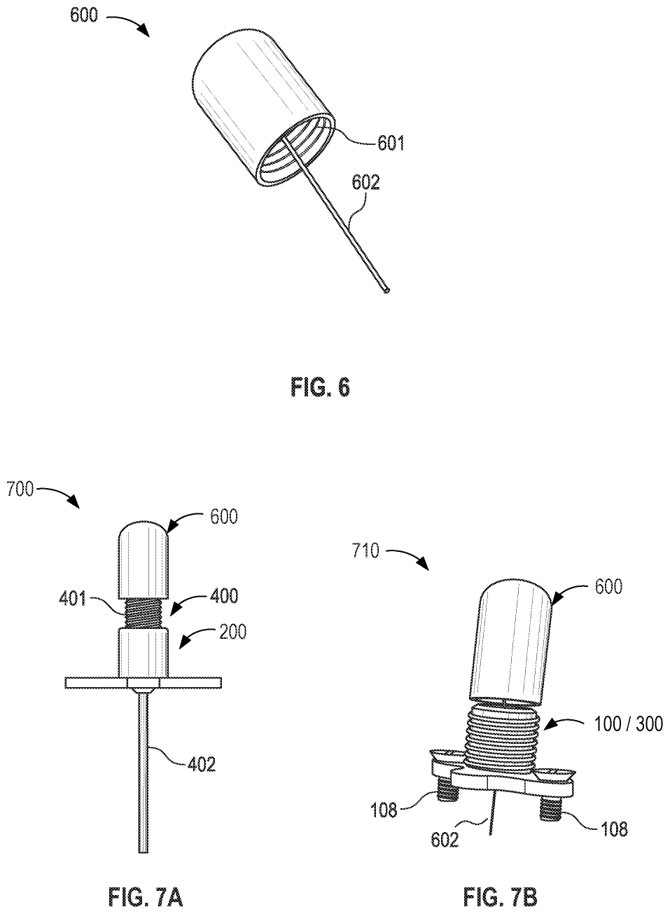

An alternate version of this threaded guide cannula (not shown) may comprise a capped version, similar in many aspects to the cannula cap 600, shown in FIG. 6 and described below, wherein the precision threads are internal to the cap, and the cannula 402 protrudes through the top of the cap, providing access to the thru-hole of the cannula. This alternate version would be configured to mate with the external threads 104 on the protrusion 101 of the first version of the precision guide 100. A similar alternate version may be provided with a solid cannula as described in the prior section above.

As illustrated in FIGS. 5A-5C, the precision guide 200 and threaded guide cannula 400 are configured for precision assembly 500 such that the user may precisely screw the cannula 402 up or down into the threaded guide 200 to a precision depth into the brain of a subject (animal), usually under fluoroscopic guidance, once the threaded guide is affixed to the scull and an opening in the scull has been prepared. FIGS. 5A-5C illustrate a representative range of penetration depths that can be achieved with the assembly. One of skill in the art would recognize that the lengths of the cannulas provided on a threaded guide cannula may be varied to allow for various ranges of penetration, depending on the size of the subject brain being treated.

At this point of a procedure, the user can then utilize a surgical micrometer (not shown) to verify and record depth or utilize a microsyringe (not shown) to take fluid and/or tissue samples from the brain through the cannula, at the subject penetration depth.

It should be noted that the microsyringe being described herein is a specialized type of syringe, sometimes referred to as a Neuroscience Injection Syringe, (AKA: Neuros). One such syringe style can be acquired from the Hamilton Company. (Neuroscience Injection Syringes (hamiltoncompany.com). In many cases these syringes may be custom built for specific applications in life science medical art specialties such as veterinary medicine, among others, where the brain of an animal (i.e.: mouse or rat) is very small, often requiring a 30-32 gauge or smaller needle and lengths of approximately 70 mm or more. Since a 70 mm long 32 gauge needle would be too flexible, it is constructed with an adjustable sleeve that maintains the needle's straightness and enables an adjustable penetration depth between 0 and 20 mm. This sleeve keeps the needle perfectly straight and provides support during insertion. These syringes typically dispense volumes between 50 nL and 100 nL through an ultrafine needle. Developed specifically for neuroscience applications, the Hamilton Neuros enables the delivery of microvolumes to an exact location while minimizing injection site damage. The Hamilton Neuros syringes come with two types of protective needle sleeves. The sleeve, with a blind stop is intended for cannulated applications and ensures targeted administration with an adjustable penetration depth. The version without a blind stop is intended for use with stereotaxic holders. Both models provide an adjustable needle exposure of 0 to 20 mm.

Additionally, as noted above, a depth micrometer may be utilized to measure the depth of penetration of the electrode or cannula, between the skull and the top of the electrode or cannula, for example. Alternately, the threaded barrels of the electrode or the cannula may have precision markings to indicate depth of penetration. Or further still, the clinician may simply utilize x-ray or fluoroscopy, often in combination with a micrometer reading or precision barrel marking, to verify depth of penetration when inserting these instruments through the Microdrive Plate into a brain or other sensitive neuro tissue.

Referring now to FIG. 6, is a representative example of a threaded cannula cap 600, configured to function with threaded cannula 400 or with precision guides 100, 200 or 300. The threaded cannula cap 600 comprises internal threads 601 and a cannula clearance stem 602 configured to keep the internal diameter of the threaded cannula 402 open or to clear any clogs that may have formed in the cannula 402 between procedures. Multiple sizes of cap 600 may be fabricated to work with either the precision guide 200 or the threaded guide cannula 400. The primary purpose of the cannula cap is to cover and seal the hole of the cannula 402 in the skull without removing the precision guide 200 and/or the threaded guide cannula 400. Depending on the internal diameter and thread of the cannula cap selected, it can be utilized with either the second version of the precision guide 200 and the threaded guide cannula 400 or the first or third version of the precision guide 100, 300 as shown if FIGS. 7A & 7B.

As illustrated in FIG. 7A, the threaded cap 600 is illustrated as it would be applied to the threaded cannula guide 400 (inserted into version two of the precision guide plate 200) creating a “capped” threaded cannula assembly 700. FIG. 7B illustrates the threaded cap 600, as it would be applied to the first version 100 or the third version 300 of the precision nut plate guide, after the threaded cannula guide 400 has been removed, creating a “capped” precision guide assembly 710. Of course, the threaded caps would be of different diameters and thread sizes for each application.

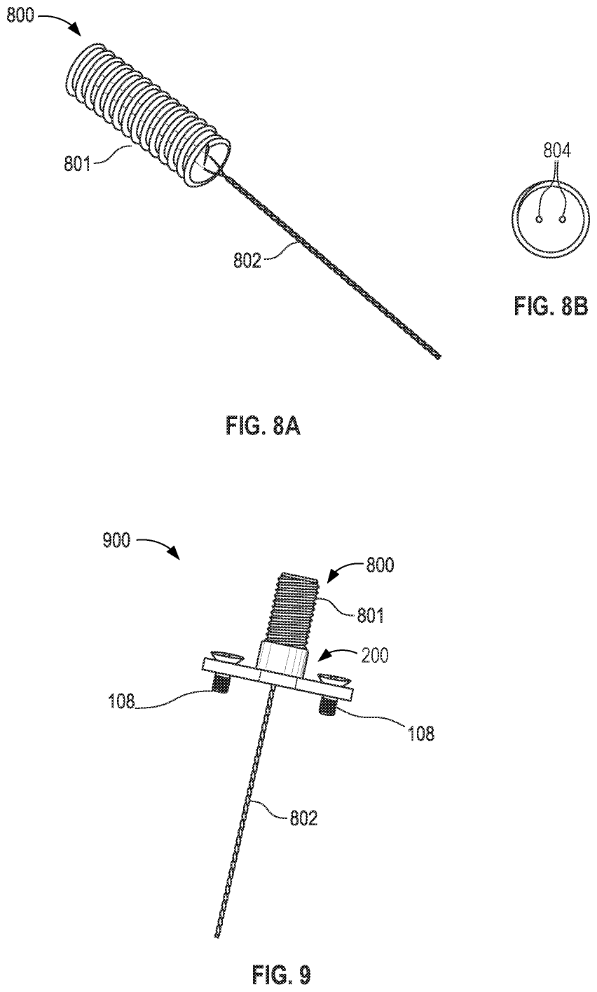

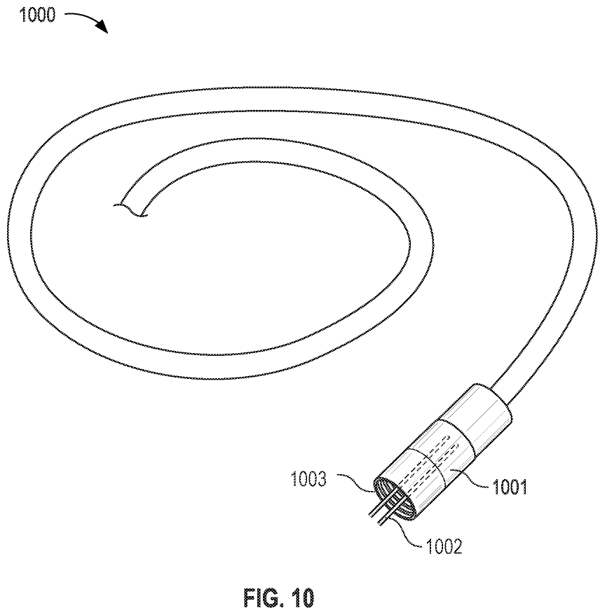

Referring now to FIG. 8A, is a representative example of a customized electrode 800 comprising a proximal threaded diameter 801 and a wire electrode 802 that extends all the way through the proximal threaded diameter 801, terminating at the proximal end of the threaded diameter to terminals 804, as shown in FIG. 8B, and configured to receive a mating connector of an electrode cable connector terminal, 1002, as illustrated in FIG. 10. This version of electrode 800 is configured to thread into either version 2 (200) or version 3 (300) of the nut plate guide.

In an alternate configuration (not shown), a customized electrode is configurable to have a cap configuration, similar to FIG. 6, but with a much longer internally threaded cap and an electrode extending therefrom. In addition, the proximal end of the cap may be threaded externally and would also have terminals 804, as shown in FIG. 8B, configured to receive a mating connector of an electrode cable connector terminal, 1002, as illustrated in FIG. 10. This alternate configuration of customized electrode would be configured to mate with nut plate guide 100, and theoretically with nut plate guide 300 as well.

Referring now to FIG. 9, is a guide assembly with electrode 900 comprising the electrode 800 threaded into the nut plate guide 200, with the electrode wire 802 extending therefrom. (Nut plate guide 300 is also an alternate configuration for this assembly). The baseplate itself, once again illustrates two or more fixation features such as screws 108 inserted and used to affix the base plate of the precision nut guide to the prepared scull of a subject.

Finally, referring now to FIG. 10, is a representative electrode cable 1000, configured to adapt on one end to an amplifier (not shown) for measuring and recording the small (<1 mV) changes in current or voltage in the subject tissue and the electrode connector cap 1001, with connector threads 1003 and the electrode cable terminal 1002 extending therefrom for connection to the electrode 800 via the electrode terminals 804.

From a procedural standpoint, the installation of the precision guide is relatively simple, involving approximately four steps; namely:

### Step 1:

Place Micro Guide on the prepared area of skull;
Mark and drill the three holes—One for the Guide and two for the Guide tab screws.

### Step 2

Add a drop of Loctite to the threaded part of the Guide Cannula,

### Step 3

Screw the Guide with Dummy Cannula into the Micro-Guide and adjust to the desired depth below the pedestal. Let set for 30 minutes so the Loctite can dry.

### Step 4

Deposit dental cement (glue) to the bottom of the Micro Guide and place assembly on the skull, Screw the tab set screws into the skull and let dry for thirty minutes.

Once installed on the veterinary subject, the Guide holds the guide cannula and allows the user to adjust the tubing below the protrusion and plate (aka pedestal) to the desired length. The guide cannula can be easily precisely adjusted within 1 mm-3 mm increments and the guide cannulas lengths are offered in 2 mm, 5 mm, 8 mm and 11 mm.

The electrodes may be offered in a variety of styles and lengths including single channel and multi-channel electrodes and the electrodes themselves can be offered in a corresponding number of lengths ranging from 5 mm to 45 mm. Mating cables can be offered in a variety of styles from single channel to multi-channel electrode systems.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A precision medical instrument guide system comprising:
a base plate having a vertical oriented protrusion thereon with a port extending through the protrusion and plate;
a guide cannula;
an electrode;
a dummy cannula; and
a port plug.

2. The precision medical instrument guide system of claim 1, further comprises:
a microsyringe needle; and
a micrometer or other measuring instrument.

3. The precision medical instrument guide system of claim 1, further comprises two or more fixation features on the base plate comprising:
holes;
screws;
nails;
pins;
spikes;
protrusions; or
a combination thereof;
wherein said fixation features are configured to provide for fixation of the base plate to a subject's boney anatomy.

4. The precision medical instrument guide system of claim 1, further comprising precision threads located on:
the protrusion;
the guide cannula;
the electrode;
the dummy cannula; and
the port plug;
configured to provide precision depth adjustment.

5. The precision medical instrument guide system of claim 4, wherein said precision threads are located on:
an exterior diameter of any component of the system;
an interior diameter of any component of the system; or
both an interior and exterior diameter of any component of the system.

6. The precision medical instrument guide system of claim 5, wherein said precision threads comprise:
an imperial fine thread series;
a metric fine or extra fine thread series;
an AJS Series;
a Thury metric screw thread;
a Löwenherz thread;
a coarse thread series; or
a combination thereof.

7. The precision medical instrument guide system of claim 1, wherein said base plate is further configured for fixation to a subject's boney anatomy with a medical grade adhesive.

8. The precision medical instrument guide system of claim 6, wherein an inferior surface of the baseplate is either:
flat; or
comprises a concave surface configured to closely conform to a boney anatomy of a skull.

9. The precision medical instrument guide system of claim 1, wherein said components can be manufactured using a variety of processes comprising:
machining;
welding,
injection molding;
casting;
additive manufacturing; or
combinations thereof.

10. The precision medical instrument guide system of claim 1, wherein said components can be manufactured using a variety of biocompatible medical grade materials comprising:
stainless steels;
polytetrafluoroethylene (PTFE)/Teflon™;
thermoplastics;
thermosets;
elastomers; or
a combination thereof.

11. The precision medical instrument guide system of claim 1, wherein said baseplate material comprises elastic behavior and plastic deformation properties that are to the left of the materials fracture point on a stress/strain curve, where strain is on the x-axis, when the baseplate is flexed to conform to the boney anatomy of a skull.

12. A precision medical instrument guide comprising:
a base plate having a port therethrough;
a vertically oriented protrusion configured to extend from the base plate;
two or more fixation features spaced about the base plate;
a port extending through the protrusion, configured to align with the port of the plate; and
threads located on the protrusion configured to receive mating threads from another instrument configured to adapt to the guide.

13. The precision medical instrument guide of claim 12, wherein the threads are:
located within the port of the protrusion;
located on the exterior of the protrusion; or
located both within the port and on the exterior of the protrusion.

14. The precision medical instrument guide of claim 12, wherein the two or more fixation features comprise:
holes;
screws;
nails;
pins;
spikes;
protrusions; or
a combination thereof;
wherein said fixation features are configured to provide for fixation of the base plate to a subject's boney anatomy.

15. The precision medical instrument guide of claim 12, wherein said base plate is further configured for attachment to a subject's boney anatomy with a medical grade adhesive.

16. The precision medical instrument guide of claim 12, wherein the base plate and the protrusion are configured:
as a single unit; or
as a multi-piece assembly.

17. The precision medical instrument guide of claim 13, wherein said precision threads are configured to receive attachments comprising:
a threaded guide cannula;
a threaded electrode;
a threaded dummy cannula; or
a threaded port plug.

18. The precision medical instrument guide of claim 13, wherein said precision threads are further configured to receive attachments comprising:
a microsyringe needle; and
a micrometer or other measuring instrument.

19. The precision medical instrument guide of claim 13, wherein said precision threads comprise:
an imperial fine thread series;
a metric fine or extra fine thread series;
an AJS thread series:
a Thury metric screw thread series;
a Löwenherz thread series;
a coarse thread series, or
a combination thereof.

20. The precision medical instrument guide of claim 14, wherein said instrument guide is configured for use in life science medical arts.

21. A precision medical instrument system comprising:
a base plate having a vertical protrusion thereon with a port extending through the protrusion and plate;
a guide cannula;
an electrode;
a dummy cannula; and
a port plug;
wherein said system is configured for intracerebral infusion or neuro-electrophysiology procedures where microsurgical work is performed.

22. The precision medical instrument system of claim 21, further comprising two or more fixation features spaced about the base plate to provide for fixation of the base plate to a subject's boney anatomy.

23. The precision medical instrument system of claim 21, further comprising threads located on:
the protrusion;
the guide cannula;
the electrode;
the dummy cannula; and
the port plug;
wherein said threads are configured to provide precision depth adjustment.

24. The precision medical instrument system of claim 23, wherein said threads are located on:
an exterior diameter of any component of the system;
an interior diameter of any component of the system; or
both an interior and exterior diameter of any component of the system.

25. The precision medical instrument system of claim 23, wherein said threads comprise:
an imperial fine thread series;
a metric fine or extra fine thread series;
an AJS thread series:
a Thury metric screw thread series;
a Löwenherz thread series;
a coarse thread series, or
a combination thereof.

26. The precision medical instrument system of claim 21, wherein said base plate is further configured for attachment to a subject's boney anatomy with a medical grade adhesive for additional fixation.

27. The precision medical instrument system of claim 21, wherein an inferior surface of the baseplate is either:
flat; or
comprises a concave surface configured to closely conform to a boney anatomy of a skull.

28. The precision medical instrument system of claim 21, wherein said components can be manufactured using a variety of processes comprising:
machining;
welding,
injection molding;
casting;
additive manufacturing; or
combinations thereof.

29. The precision medical instrument system of claim 21, wherein said components can be manufactured using a variety of biocompatible medical grade materials comprising:
stainless steels;
polytetrafluoroethylene (PTFE)/Teflon™;
thermoplastics;
thermosets;
elastomers; or
a combination thereof.

30. The precision medical instrument system of claim 21, wherein said baseplate material comprises elastic behavior and plastic deformation properties that are to the left of the materials fracture point on a stress/strain curve, where strain is on the x-axis, when the baseplate is flexed to conform to the boney anatomy of a skull.
